(19) United States
(12) Patent Application Publication
(10) Pub. No.: US 2026/0325267 A1
(43) Pub. Date: Oct. 22, 2026

(54) AN INTRAORAL SCANNING DEVICE WITH A HYGIENIC IMPLEMENTATION OF LEDS

(71) Applicant: 3SHAPE A/S, Copenhagen K (DK)

(72) Inventors: Anders LARSEN, Copenhagen K (DK); Alexander Bruun CHRISTIANSEN, Copenhagen K (DK); Kasper Krogh HANSEN, Copenhagen K (DK)

(73) Assignee: 3SHAPE A/S, Copenhagen K (DK)

(21) Appl. No.: 19/106,893

(22) PCT Filed: Aug. 21, 2023
(86) PCT No.: PCT/EP2023/072936

§ 371 Date: Feb. 26, 2025

(30) Foreign Application Priority Data
Aug. 29, 2022 (EP) 22192586.0
Sep. 13, 2022 (EP) 22195268.2

(51) Int. Cl. A61C 9/00

(52) U.S. Cl. CPC A61C 9/0053; A61C 2204/002

(57) ABSTRACT

An intraoral scanning device includes a processing unit configured to process intraoral scan data of a patient and provide 2D image data and/or 3D image data; a wireless interface configured to transmit the 2D image data and/or the 3D image data; a battery configured to power at least the processing unit and the wireless interface, and a housing configured to accommodate at least the processing unit, the wireless interface and at least partially the battery. The intraoral scanning device may further include a plurality of LEDs accommodated by the housing. The plurality of LEDs may be configured to emit light through a transparent part of the housing made of a material that is configured emit the light that passes through the material. The emitted light may indicate a status of the battery and/or the wireless interface, and/or indicate a mode of the intraoral scanning device.

### FIELD

The disclosure relates to a hygienic implementation of status LEDs in an intraoral scanning device.

### BACKGROUND

In the field of dentistry, it is of great importance that medical devices meet national and international standards for hygiene to ensure safety of the patient and to ensure safe use of the devices.

This generally implies that medical devices that are in the immediate vicinity of a patient have surfaces that, as much as possible, reduce the risk of contamination. Surfaces having cavities, even relatively small cavities, such as grooves, may allow fluids from a patient, such as saliva and blood, to reach these cavities, and may allow bacteria to thrive inside them and lead to contamination.

Cavities and grooves in the surface of medical devices will further complicate the process of cleaning the devices, and therefore leads to a further risk of spreading the bacteria to other patients. Such grooves are usually present in the housing or casing of medical devices, particularly at portions, where different parts of the housing or casing meet.

### SUMMARY

An aspect of the present disclosure is to improve hygiene of intraoral scan devices and to reduce the risk of contamination of a surface of the housing of intraoral scan devices.

According to the aspect, an intraoral scanning device is disclosed. The intraoral scanning device may include a processing unit configured to process intraoral scan data of a patient and provide 2D image data and/or 3D image data; a wireless interface configured to transmit the 2D image data and/or the 3D image data; a battery configured to power at least the processing unit and the wireless interface, and a housing configured to accommodate at least the processing unit, the wireless interface and at least partially the battery.

The intraoral scanning device may further include a plurality of LEDs accommodated by the housing. The plurality of LEDs may be configured to emit light through a transparent part of the housing made of a material that is configured emit the light that passes through the material. The emitted light may indicate a status of the battery and/or the wireless interface, and/or indicate a mode of the intraoral scanning device.

Allowing the emitted light to penetrate a transparent part of the housing results in an implementation where the plurality of LEDs is not arranged on the housing or in a groove of the housing, resulting in a smooth outer surface of the housing which is easy to clean. If the plurality of LEDs was arranged on the housing, then dirt will be gathered at the surfaces of each of the plurality of LEDs or at surfaces of a structure that comprises the plurality of LEDs. Corners will be created by placing the plurality of LEDs on the housing which will be difficult to clean. In the other situation where the housing includes one or more grooves for the plurality of LEDS, the dirt will go into the one or more grooves, and unfortunately, most of the time will be caught between the housing and each of the plurality of LEDs in the one or more grooves.

The material of the transparent part may be configured to diffuse the emitted light that passes through the material. The diffusion provides a more aesthetic look and a visual effect that makes it more comfortable to look at. If no diffusion of the emitted light occurs, then a light pattern of a one or more of the plurality of LEDs may disturb the eyes of a user of the intraoral scanning device or the patient being scanned.

The diffusion of the emitted light from the plurality of LEDs happens inside the plastic shell of the transparent part, wherein the light is being scattered until eventually reaching the transparent coating. The diffusion of the emitted light will appear as a homogeneous light pattern.

The plurality of LEDs is arranged to have a certain distance between each of the LEDs and with a certain distance between each of the LEDS and the inner surface of the housing so that an optimal diffusion of the emitted light will appear. An optimal diffusion would have no hot spots where a light patten from a single LED is seen.

The transparent part of the housing may be made of at least a single layered coating structure, and at least another part of the housing may be made of a multiple layered coating structure, and wherein an outer layer of the multiple layered coating and the single layered coating structure comprise the same material. The outer layer of the multiple layered coating corresponds to the outer layer of the housing. The single layered coating structure corresponds to the outer layer of the housing. During a coating process of the housing the single layered coating structure and the outer layer of the multiple layered coating structure are coated onto the housing in a single coating process. The housing may comprise a plastic shell which the coatings are coated onto.

The housing may comprise a plastic shell which is then coated with the multi layered coating structure and the single layered coating structure.

The shell may be made of opal white Polycarbonate, which is then coated with the single layered coating structure and the multiple layered coating structure. The multiple layered coating structure includes following layers:
a first layer including a base coating that minimizes light going into the second layer,
a second layer including a coloured layer, and
a third layer including a transparent coating.

The single layered coating structure may include a transparent coating. The transparent coating of both the single layered coating structure and the multiple layered coating structure may be the same.

The transparent coating of both the single layered coating structure and the multiple layered coating structure may be coated on at the same time and in a single coating process.

The first layer may include at least white and/or silver particles for scattering light that penetrates the plastic shell away from the second layer.

By coating the single layered coating structure and the outer layer of the multiple layered coating structure results in a uniform outer surface of the housing is achieved where dirt can easily be cleaned away.

The outer surface of the transparent part may be about aligned with an outer surface of the at least another part of the housing.

The plurality of LEDs may be arranged circumferentially to the housing. In other words, the plurality of LEDs is arranged circumferentially relative to an inner surface of the housing or the plastic shell of the housing. The plurality of LEDs may be arranged on a ring structure that is connected to an inner module that comprises electronics and/or optics of the intraoral scanning device. The ring structure may go around the inner module and follow the inner surface of the housing. The plurality of LEDs may be arranged such that each LED has approximately the same distance to the inner surface of the housing. The ring structure may be a printed circuit board. The ring structure may comprise a groove in which the plurality of LEDs may be arranged. The side walls of the groove may include a light blocker configured to block the emitted light from the plurality of LEDs to emerge sideways. Thereby, the emitted light is focused upwards and towards the outer surface of the housing, i.e., the transparent coating layer.

Each of the plurality of LEDs may have a lighting axis that penetrate the inner surface of the housing with an angle. The angle between the inner surface and the lighting axis may be between 45 degrees and 90 degrees.

The plurality of LED includes at least three different colour spectra, such as white, blue or green. The colours can be any colours suitable for the purpose of the plurality of LEDs.

The processing unit may be configured to generate at least one control signal to control power delivered to one or more of the plurality of LEDs.

The processing unit may include one or more processors, wherein the one or more processors are configured to process the intraoral scan data and to control the plurality of LEDs.

The processing unit may be further configured to change at least one control signal over time so that the plurality of LEDs emits at least one static or dynamic lighting effect.

The processing unit may be configured to receive at least one control signal from the wireless interface, and wherein the at least one control signal includes information about the status of the wireless interface. The status of the wireless interface may be one or more of the following:
a connecting status indicating that the wireless interface is connecting to an external device,
a connect status indicating that the wireless interface is connected to the external device, and
a failed to connect status indicating that the wireless interface failed to connect to the external device.

For example, when the status is the “connecting status” then the plurality of LEDS provides a rotational dynamic lighting effect with a single colour of spectra. For example, each of the plurality of LEDs blinks such that a rotational appearance of the emitted light is visualized. The single colour may be blue, green or white. In another example, when the status is the “connect status” then the plurality of LEDs emits lights such that the appearance of the emitted light from the plurality of LEDs seems solid or constant. In yet another example, when the status may be the “failed to connect” the plurality of LEDs blinks such that a static appearance of the emitted light is visualized. The blinking of the LEDs may appear at least three times. A vibrator may be arranged within the intraoral scanner, and the vibrator may be initiated along with the blinking of the LEDs when the status is the “failed to connect”.

The processing unit may receive the at least one control signal from a power management unit of the intraoral scanning device, and the at least one control signal includes information about the status of the battery. The status of the battery may be a low battery power status indicating that the battery should be recharged or exchanges with a new battery. In one example, when the status may be the “failed to connect” the plurality of LEDs blinks such that a static appearance of the emitted light is visualized. The blinking of the LEDs may appear at least three times. The blinking in this example may be of a different colour than in the example where the status is the “failed to connect”. A vibrator may be arranged within the intraoral scanner, and the vibrator may be initiated along with the blinking of the LEDs when the status is the “failed to connect”.

The processing unit may be configured to receive the at least one control signal from the processing unit and/or a power management unit of the intraoral scanning device, and the at least one control signal includes information about the mode of the intraoral scanning device. The mode of the intraoral scanning device may be one or more of the following:
an idle mode indicating that the intraoral scanning device is ready to perform a scan,
a sleeping mode indicating that the intraoral scanning device is operating with a lower power consumption than in the idle mode,
a start scan mode indicating that the intraoral scanning device is starting a scan session,
a scanning mode indicating that the intraoral scanning device is in a scan session, and
a registration lost mode indicating that the intraoral scanning device failed to process intraoral scan data captured by an image sensor of the intraoral scanning device.

The plurality of LEDS may be arranged in the vicinity of a tip cover of the intraoral scanner device. The tip cover is configured to be detachable mounted onto the intraoral scanning device for protecting a scanner tip of the intraoral scanning device. The scanner tip is configured to guide the light being transmitted from a projector unit onto a dental object. When the tip cover is mounted onto the scanner tip, the emitted light may pass through a window that is made of glass. The plurality of LEDs may be arranged between the scanner tip and a button interface of the intraoral scanning device. In any of the arrangements of the plurality of LEDs, a clear and undisturbed view of the plurality of LEDs is achieved during the use of the intraoral scanning device.

According to the aspect, an intraoral scanning device is disclosed. The intraoral scanning device may be configured to acquire intraoral scan data from a three-dimensional dental object during a scanning session. The intraoral scanning device may include a processing unit that may be configured to process intraoral scan data of a patient and provide 2D image data and/or 3D image data. The intraoral scanning device may further include a memory. The intraoral scanning device may further include a wireless interface that may be configured to transmit 2D image data and/or 3D image data. The intraoral scanning device may further include a plurality of LEDs. The intraoral scanning device may further include a battery configured to power at least the processing unit, the wireless interface, and the plurality of LEDs. The intraoral scanning device may further include a housing that may be configured to enclose the processing unit, the memory, the wireless interface, the plurality of LEDs, and at least partly enclosing the battery. The housing may be made of a material that is configured to diffuse LED light passing through the material. A part of the housing may include a coating that is configured to block the propagation of LED light.

According to the aspect, an intraoral scanning device is disclosed. The intraoral scanning device may be configured to acquire intraoral scan data from a three-dimensional dental object during a scanning session. The intraoral scanning device may include a processing unit that may be configured to process intraoral scan data of a patient and provide 2D image data and/or 3D image data. The intraoral scanning device may further include a memory. The intraoral scanning device may further include a wireless interface that may be configured to transmit 2D image data and/or 3D image data. The intraoral scanning device may further include a plurality of LEDs. The intraoral scanning device may further include a battery configured to power at least the processing unit, the wireless interface, and the plurality of LEDs. The intraoral scanning device may further include a housing that may be configured to enclose the processing unit, the memory, the wireless interface, the plurality of LEDs, and at least partly enclosing the battery. The housing may be made of a material that is configured to scatter LED light passing through the material. A part of the housing may include a coating that is configured to block the propagation of LED light.

According to the aspect, an intraoral scanning device is disclosed. The intraoral scanning device may be configured to acquire intraoral scan data from a three-dimensional dental object during a scanning session. The intraoral scanning device may include a processing unit that may be configured to process intraoral scan data of a patient and provide 2D image data and/or 3D image data. The intraoral scanning device may further include a memory. The intraoral scanning device may further include a wireless interface that may be configured to transmit 2D image data and/or 3D image data. The intraoral scanning device may further include a plurality of LEDs. The intraoral scanning device may further include a battery configured to power at least the processing unit, the wireless interface, and the plurality of LEDs. The intraoral scanning device may further include a housing that may be configured to enclose the processing unit, the memory, the wireless interface, the plurality of LEDs, and at least partly enclosing the battery. The housing may be made of a material that is configured to distribute LED light passing through the material evenly. A part of the housing may include a coating that is configured to block the propagation of LED light.

The coating may be a painting that is configured to prevent LED light from passing through the painting.

The plurality of LEDs may further be enclosed by the part of the housing not including the coating or painting.

The plurality of LEDs may further be arranged below the part of the housing not including the coating or painting.

The plurality of LEDs may further be configured to emit light in different colours and wavelengths, and may further be configured to blink in different patterns, for example by reducing, enhancing, and completely cutting off the power supply to some or all of them.

The plurality of LEDs may be configured to emit light in a colour and/or wavelength and/or pattern in response to a received control signal.

The control signal may be generated based on a status or mode of the intraoral scanning device or based on a user input, as will be described further below in this summary section.

The housing may be manufactured by injection moulding of a hollow shell in a polycarbonate material. The polycarbonate material may be configured to diffuse light passing through the material. The polycarbonate may be a white polycarbonate.

The transparent part of the housing may be created by covering a part of an outer surface of the housing and applying a coating on another part of the outer surface of the housing. The coating may be a painting that is configured to prevent light to pass through the painting, such as a mix of white and silver painting. Covering a part of the housing may result in that particular part not being covered by the coating, and thereby may allow light to pass through that particular part of the housing, and that particular part may thus stay transparent.

The housing may further be coated by a second coating, that may be applied on top of the outer surface of the housing on the transparent part of the housing and on the another part of the housing. The second coating may be configured to level out the surface of the housing and fill in the space that may be present as a result of difference in hight between the transparent part of the housing and the another part of the housing. Levelling out the difference in hights on the outer surface of the housing may lead to a more smooth surface and may lead to reducing edges and/or grooves, and may therefore reduce the risk of gathering of bacteria at such edges and/or grooves, and may thus result in a more hygienic housing.

The intraoral scanning device may further comprise a user interface that may be adapted to receive a user input to control operation of the processing unit so as to facilitate a change in the colour that is a combination of the at least three different colour spectra. The user interface may be a part of a user interface that is configured to control scanning operations of the intraoral scanning device or may be a separate user interface that may be dedicated to receive the user input to control operation of the processing unit so as to facilitate a change in the colour, such as for example a touch panel in the vicinity of the transparent area.

### BRIEF DESCRIPTION OF THE FIGURES

Aspects of the disclosure may be best understood from the following detailed description taken in conjunction with the accompanying figures. The figures are schematic and simplified for clarity, and they just show details to improve the understanding of the claims, while other details are left out. Throughout, the same reference numerals are used for identical or corresponding parts. The individual features of each aspect may each be combined with any or all features of the other aspects. These and other aspects, features and/or technical effect will be apparent from and elucidated with reference to the illustrations described hereinafter in which:

FIG. 1 illustrates an intraoral scanning device comprising a housing accommodating a plurality of LEDs;

FIG. 2 illustrates the intraoral scanning device comprising a transparent part of the housing covering the plurality of LEDs;

FIG. 3 schematically shows a cross-sectional view of the housing and schematically illustrating the scattering of the LED light inside a side wall of the housing;

FIGS. 4A and 4B schematically illustrate the plurality of LEDs circumferentially distributed along an inner surface of the housing;

FIG. 5 illustrates an intraoral scanning device comprising a housing accommodating a plurality of LEDs;

FIG. 6 illustrates a light guiding path in the housing;

FIG. 7 illustrates the intraoral scanning device comprising a power management unit according to an aspect;

FIG. 8 schematically shows a cross-sectional view of the housing and schematically illustrates the scattering of the LED light inside a side wall of the housing according to an aspect; and

FIG. 9 shows a table with an overview of control signals from the wireless interface and the power management unit and examples of resulting visual effects performed by the plurality of LEDs.

### DETAILED DESCRIPTION

The detailed description set forth below in connection with the appended drawings is intended as a description of various configurations. The detailed description includes specific details for the purpose of providing a thorough understanding of various concepts. However, it will be apparent to those skilled in the art that these concepts may be practiced without these specific details. Several aspects of the devices, systems, mediums, programs and methods are described by various blocks, functional units, modules, components, circuits, steps, processes, algorithms, etc. (collectively referred to as “elements”). Depending upon particular application, design constraints or other reasons, these elements may be implemented using electronic hardware, computer program, or any combination thereof.

The electronic hardware may include microprocessors, microcontrollers, digital signal processors (DSPs), field programmable gate arrays (FPGAs), programmable logic devices (PLDs), gated logic, discrete hardware circuits, and other suitable hardware configured to perform the various functionality described throughout this disclosure. Computer program shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise.

A scanning for providing extra-oral scan data and/or intra-oral scan data may be performed by a dental scanning system that may include an intraoral scanning device such as the TRIOS series scanners from 3Shape A/S or a laboratory-based scanner such as the E-series scanners from 3Shape A/S. The dental scanning system may include a wireless capability as provided by a wireless network unit. The scanning device may employ a scanning principle such as triangulation-based scanning, confocal scanning, focus scanning, ultrasound scanning, x-ray scanning, stereo vision, structure from motion, optical coherent tomography OCT, or any other scanning principle. In an embodiment, the scanning device is capable of obtaining surface information by operated by projecting a pattern and translating a focus plane along an optical axis of the scanning device and capturing a plurality of 2D images at different focus plane positions such that each series of captured 2D images corresponding to each focus plane forms a stack of 2D images. The acquired 2D images are also referred to herein as raw 2D images, wherein raw in this context means that the images have not been subject to image processing. The focus plane position is preferably shifted along the optical axis of the scanning system, such that 2D images captured at a number of focus plane positions along the optical axis form said stack of 2D images (also referred to herein as a sub-scan) for a given view of the object, i.e., for a given arrangement of the scanning system relative to the object. After moving the scanning device relative to the object or imaging the object at a different view, a new stack of 2D images for that view may be captured. The focus plane position may be varied by means of at least one focus element, e.g., a moving focus lens. The scanning device is generally moved and angled relative to the dentition during a scanning session, such that at least some sets of sub-scans overlap at least partially, in order to enable reconstruction of the digital dental 3D model by stitching overlapping subscans together in real-time and display the progress of the virtual 3D model on a display as feedback to the user. The result of stitching is the digital 3D representation of a surface larger than that which can be captured by a single sub-scan, i.e., which is larger than the field of view of the 3D scanning device. Stitching, also known as registration and fusion, works by identifying overlapping regions of 3D surface in various sub-scans and transforming sub-scans to a common coordinate system such that the overlapping regions match, finally yielding the digital 3D model. An Iterative Closest Point (ICP) algorithm may be used for this purpose. Another example of a scanning device is a triangulation scanner, where a time varying pattern is projected onto the dental object and a sequence of images of the different pattern configurations are acquired by one or more cameras located at an angle relative to the projector unit.

Colour texture of the dental object may be acquired by illuminating the object using different monochromatic colours such as individual red, green and blue colours or my illuminating the object using multichromatic light such as white light. A 2D image may be acquired during a flash of white light.

Generally, the process of obtaining surface information in real time of a dental object to be scanned requires the scanning device to illuminate the surface and acquire high number of 2D images. Typically, a high-speed camera is used with a framerate of 300-2000 2D frames pr second dependent on the technology and 2D image resolution. The high amount of image data needed to be handled by the scanning device to eighter directly forward the raw image data stream to an external processing device or performing some image processing before transmitting the data to an external device or display. This process requires that multiple electronic components inside the scanner is operating with a high workload thus requiring a high demand of current.

The scanning device comprises one or more light projectors configured to generate an illumination pattern to be projected on a three-dimensional dental object during a scanning session. The light projector(s) preferably comprises a light source, a mask having a spatial pattern, and one or more lenses such as collimation lenses or projection lenses. The light source may be configured to generate light of a single wavelength or a combination of wavelengths (mono-or polychromatic). The combination of wavelengths may be produced by using a light source configured to produce light (such as white light) comprising different wavelengths. Alternatively, the light projector(s) may comprise multiple light sources such as LEDs individually producing light of different wavelengths (such as red, green, and blue) that may be combined to form light comprising the different wavelengths. Thus, the light produced by the light source may be defined by a wavelength defining a specific colour, or a range of different wavelengths defining a combination of colours such as white light. In an embodiment, the scanning device comprises a light source configured for exciting fluorescent material of the teeth to obtain fluorescence data from the dental object. Such a light source may be configured to produce a narrow range of wavelengths. In another embodiment, the light from the light source is infrared (IR) light, which is capable of penetrating dental tissue. The light projector(s) may be DLP projectors using a micro mirror array for generating a time varying pattern, or a diffractive optical element (DOF), or back-lit mask projectors, wherein the light source is placed behind a mask having a spatial pattern, whereby the light projected on the surface of the dental object is patterned. The back-lit mask projector may comprise a collimation lens for collimating the light from the light source, said collimation lens being placed between the light source and the mask. The mask may have a checkerboard pattern, such that the generated illumination pattern is a checkerboard pattern. Alternatively, the mask may feature other patterns such as lines or dots, etc.

The scanning device preferably further comprises optical components for directing the light from the light source to the surface of the dental object. The specific arrangement of the optical components depends on whether the scanning device is a focus scanning apparatus, a scanning device using triangulation, or any other type of scanning device. A focus scanning apparatus is further described in EP 2 442 720 B1 by the same applicant, which is incorporated herein in its entirety.

The light reflected from the dental object in response to the illumination of the dental object is directed, using optical components of the scanning device, towards the image sensor(s). The image sensor(s) are configured to generate a plurality of images based on the incoming light received from the illuminated dental object. The image sensor may be a high-speed image sensor such as an image sensor configured for acquiring images with exposures of less than 1/1000 second or frame rates in excess of 250 frames pr. Second (fps). As an example, the image sensor may be a rolling shutter (CCD) or global shutter sensor (CMOS). The image sensor(s) may be a monochrome sensor including a colour filter array such as a Bayer filter and/or additional filters that may be configured to substantially remove one or more colour components from the reflected light and retain only the other non-removed components prior to conversion of the reflected light into an electrical signal. For example, such additional filters may be used to remove a certain part of a white light spectrum, such as a blue component, and retain only red and green components from a signal generated in response to exciting fluorescent material of the teeth.

The network unit may be configured to connect the dental scanning system to a network comprising a plurality of network elements including at least one network element configured to receive the processed data. The network unit may include a wireless network unit or a wired network unit. The wireless network unit is configured to wirelessly connect the dental scanning system to the network comprising the plurality of network elements including the at least one network element configured to receive the processed data. The wired network unit is configured to establish a wired connection between the dental scanning system and the network comprising the plurality of network elements including the at least one network element configured to receive the processed data.

The dental scanning system preferably further comprises a processor configured to generate scan data (such as extra-oral scan data and/or intra-oral scan data) by processing the two-dimensional (2D) images acquired by the scanning device. The processor may be part of the scanning device. As an example, the processor may comprise a Field-programmable gate array (FPGA) and/or an Advanced RISC Machines (ARM) processor located on the scanning device. The scan data comprises information relating to the three-dimensional dental object. The scan data may comprise any of: 2D images, 3D point clouds, depth data, texture data, intensity data, colour data, and/or combinations thereof. As an example, the scan data may comprise one or more-point clouds, wherein each point cloud comprises a set of 3D points describing the three-dimensional dental object. As another example, the scan data may comprise images, each image comprising image data e.g., described by image coordinates and a timestamp (x, y, t), wherein depth information can be inferred from the timestamp. The image sensor(s) of the scanning device may acquire a plurality of raw 2D images of the dental object in response to illuminating said object using the one or more light projectors. The plurality of raw 2D images may also be referred to herein as a stack of 2D images. The 2D images may subsequently be provided as input to the processor, which processes the 2D images to generate scan data. The processing of the 2D images may comprise the step of determining which part of each of the 2D images are in focus in order to deduce/generate depth information from the images. The depth information may be used to generate 3D point clouds comprising a set of 3D points in space, e.g., described by cartesian coordinates (x, y, z). The 3D point clouds may be generated by the processor or by another processing unit. Each 2D/3D point may furthermore comprise a timestamp that indicates when the 2D/3D point was recorded, i.e., from which image in the stack of 2D images the point originates. The timestamp is correlated with the z-coordinate of the 3D points, i.e., the z-coordinate may be inferred from the timestamp. Accordingly, the output of the processor is the scan data, and the scan data may comprise image data and/or depth data, e.g., described by image coordinates and a timestamp (x, y, t) or alternatively described as (x, y, z). The scanning device may be configured to transmit other types of data in addition to the scan data. Examples of data include 3D information, texture information such as infra-red (IR) images, fluorescence images, reflectance colour images, x-ray images, and/or combinations thereof.

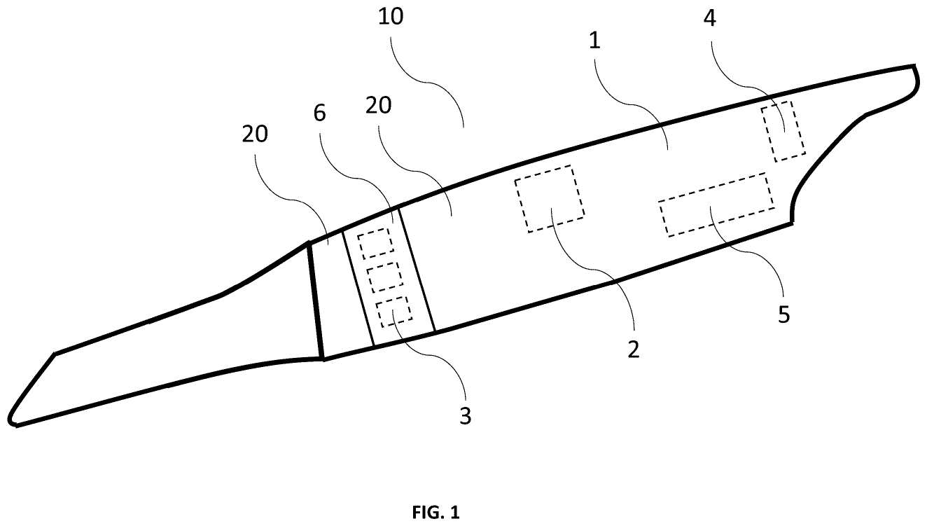

FIG. 1 illustrates an intraoral scanning device 10 for acquiring intraoral scan data from a three-dimensional dental object during a scanning session. The intraoral scanning device comprising 10 a processing unit 2 configured to process intraoral scan data of a patient and provide 2D image data and/or 3D image data, a wireless interface 4 configured to transmit the 2D image data and/or the 3D image data; a battery 5 configured to power at least the processing unit 2 and the wireless interface 4, and a housing 1 configured to accommodate at least the processing unit 2, the wireless interface 4 and at least partially the battery 5. The housing 1 further comprises a plurality of LEDs 3 and the plurality of LEDs 3 is configured to emit light through a transparent part 6 of the housing 1 made of a material that is configured to emit the light that passes through the material, and wherein the emitted light indicates a status of the battery 5 and/or the wireless interface 4, and/or indicates a mode of the intraoral scanning device 10.

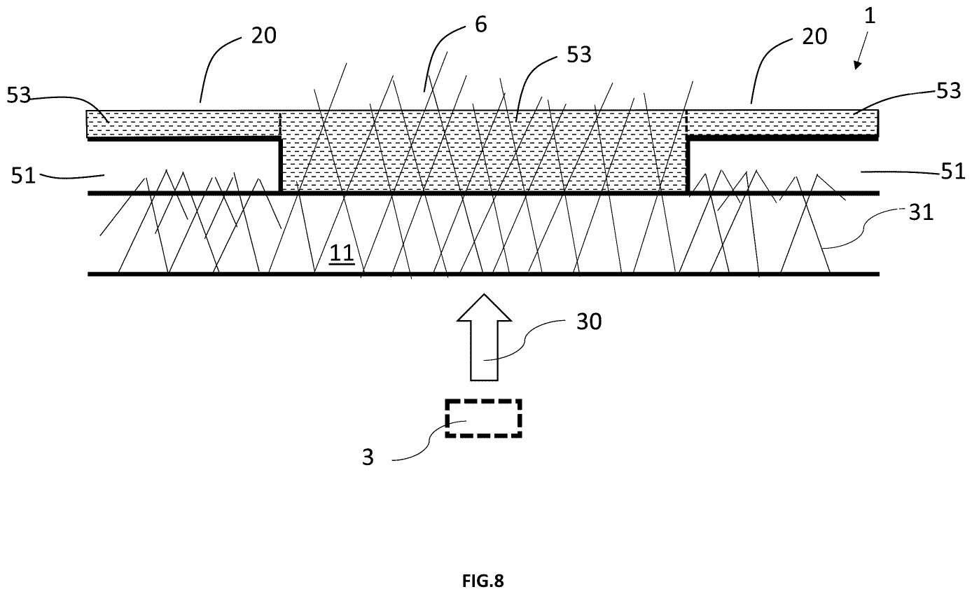

In another example, FIG. 1 illustrates an intraoral scanning device 10 according to an aspect of the disclosure, for acquiring intraoral scan data from a three-dimensional dental object during a scanning session. The intraoral scanning device 10 includes a processing unit 2 configured to process intraoral scan data of a patient and provide 2D image data and/or 3D image data, a memory, a wireless interface 4 configured to transmit 2D image data and/or 3D image data, a plurality of LEDs 3, a battery 5 configured to power at least the processing unit 2, the wireless interface 4, and the plurality of LEDs 3. The intraoral scanning device 10 further includes a housing 1 configured to enclose the processing unit 2, the memory, the wireless interface 4, the plurality of LEDs 3, and at least partly enclosing the battery 5. The housing 1 may be made of a material that is configured to diffuse LED light passing through the material, scatter LED light passing through the material, and/or distribute LED light passing through the material evenly. A part 20 of the housing 1 may include a coating 51 (shown in FIG. 8) that is configured to block the propagation of LED light. FIG. 1 further illustrates the intraoral scanning device 10 showing a transparent part 6 of the housing 1 covering the plurality of LEDs 3.

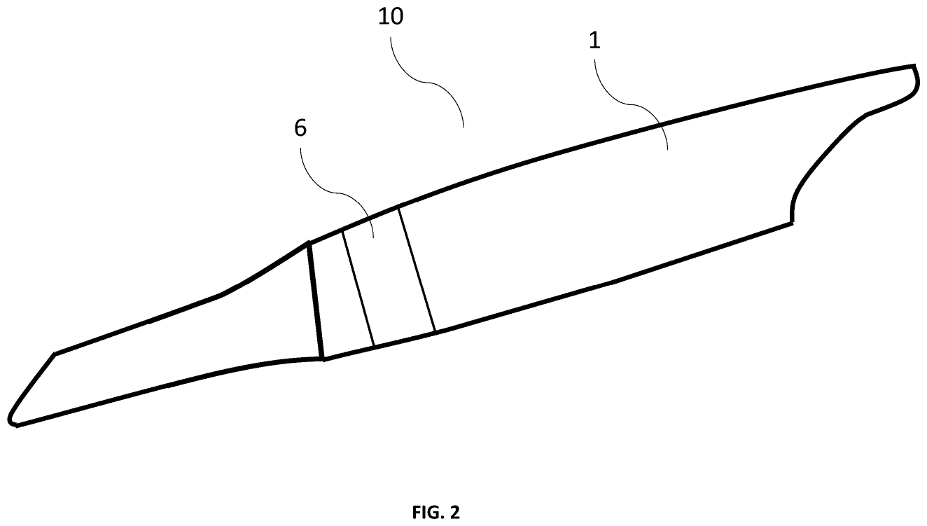

FIG. 2 illustrates the intraoral scanning device (10) of FIG. 1 showing the transparent part (6) of the housing covering the plurality of LEDs (3).

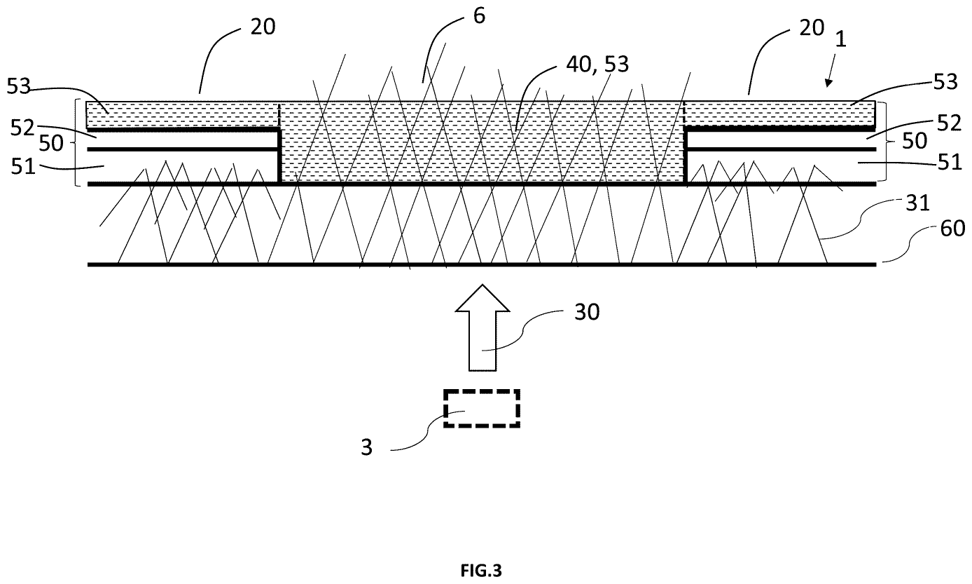

FIG. 3 illustrates an example of the housing 1 including a plastic shell 60 made of polycarbonate material that is configured to scatter light 31 passing through the material thereby diffusing the emitted light 30 from the plurality of LEDs 3. The transparent part 6 of the housing 1 includes a single layered coating structure 40 that includes a transparent coating provided directly on the polycarbonate material 60. In another part 20 of the housing 1 a multiple layered coating structure 50 is applied. The multiple layered coating structure 50 includes at least a first layer 51 that includes at least white and silver particles, a second layer 52 that includes a coloured layer and a third layer 53 which is the transparent coating similar to the transparent coating 53 provided on the housing 1 at the transparent part 6. The second layer is applied through a colouring process.

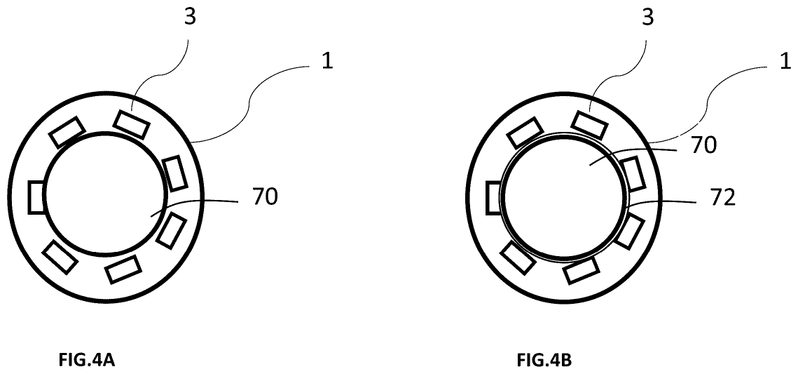

FIGS. 4A and 4B schematically illustrates a cross-sectional front view of the housing 1 showing the plurality of LEDs 3 distributed circumferentially along an inner surface of the housing (1) and accommodated therein. In this present example, the plurality of LEDs is arranged around an inner module 70 that may comprise electronics and/or optical components of the intraoral scanning device. The plurality of LEDs is directly connected to a printed circuit board that is part of the inner module 70, see FIG. 4A. In another example, the printed circuit board may be a separate component that is connected to an outer surface of the inner module, see FIG. 4B.

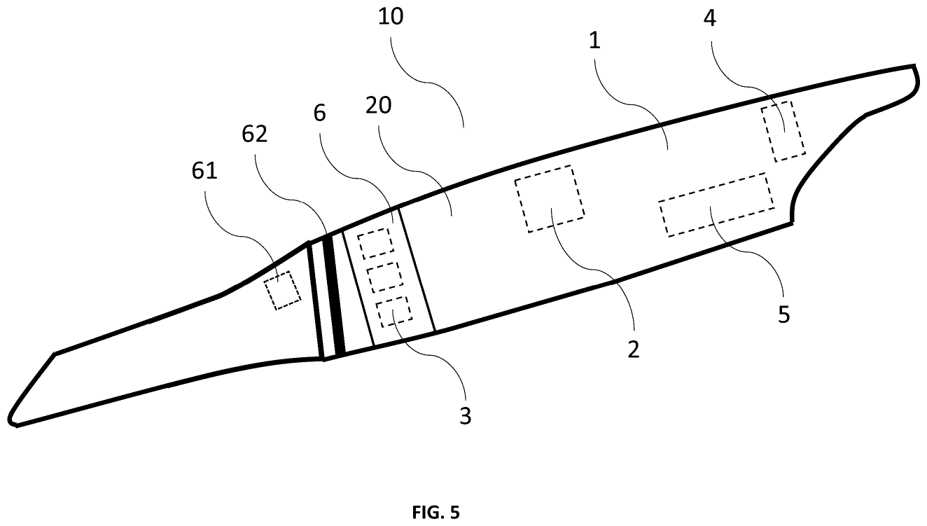

FIG. 5 illustrates the intraoral scanning device 10 that includes a sensor light blocker 62 arranged on a first side of the plurality of LEDs 3. The sensor light blocker 62 prevents the emitted light from the plurality of LEDs 3 to be captured by a tip sensor 61.

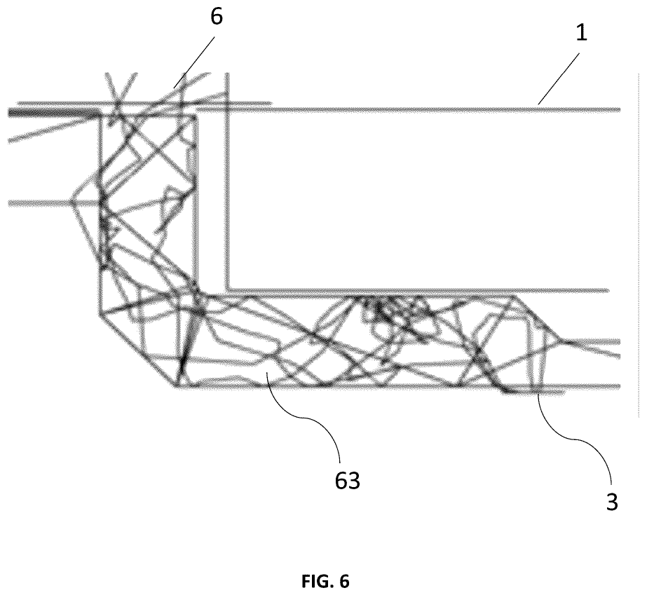

FIG. 6 illustrates an example of the housing 1 that includes a light guiding path 63 arranged in for example the plastic shell of the housing 1. In this present example, the plurality of LEDs 3 is arranged outside the transparent part 6. The emitted light from the plurality of LEDs 3 is then guided via the light guiding path 63 towards the transparent part 6. The light guiding path 63 is L shaped as the plurality of LEDs is arranged outside the transparent part 6. In another example, the light guiding path 63 may be vertically straight as the plurality of LEDs is arranged inside the transparent part 6. In this example, the light guiding path 63 would have the advantage of focusing the emitted light outwards and towards the outer surface of the housing 1.

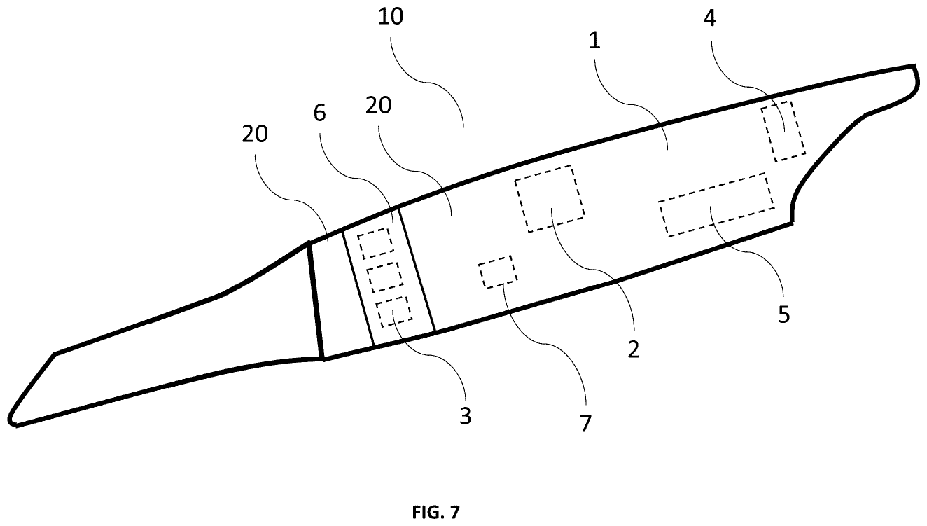

FIG. 7 shows the intraoral scanning device 10 further comprising a power management unit 7. The power management unit 7 may be a software, a physical component or a physical component controlled by a software. The power management unit 7 may be configured to control power supply to the wireless interface 4 and to the plurality of LEDs 3. The power management unit 7 may be configured to receive control signals from the processing unit 2 with information about power delivery to the wireless interface 4 and to the plurality of LEDs 3. The power management unit 7 may be configured to send and receive control signals to and from the processing unit 2, the wireless interface 4, the battery 5, and the plurality of LEDs 3.

FIG. 8 schematically illustrates a part of a cross-section of the housing 1 that covers one of the plurality of LEDs 3. The one of the plurality of LEDs 3 is shown emitting light (represented by an arrow 30) towards an inner surface of a side wall 11 of the housing 1.

The figure further illustrates the scattering (schematically illustrated by lines 31) of the emitted light from the one of the plurality of LEDs 3 inside the side wall 11 of the housing 1. Some of the scattered light 31 is shown leaving an outer surface of the side wall 11 of the housing 1 through a part 20 of the housing 1 that is covered by a coating 51. The coating 51 is shown preventing a part of the scattered light 31 from passing through the coating 51 and that part of the scattered light 31 will therefore not be visible from the outside of the housing 1. The coating 51 may be a painting, such as a mix of white and silver. Some of the scattered light 31 is shown leaving an outer surface of the side wall 11 of the housing 1 through the transparent part 6 of the housing 1. This transparent part 6 of the housing 1 is not covered by the coating 51, and the scattered light 31 will therefore be visible as a diffused and evenly distributed light at the transparent part 6 of the housing 1 from the outside of the housing 1. The figure is further showing a transparent coating 53 covering the coating 51 at the part 20 of the housing 1 that is covered by the coating 51 and covering the surface of the housing 1 at the transparent part 6 of the housing 1. The transparent coating 53 is shown levelling out the difference in hight between the part 20 of the housing that is covered by the coating 51 and the transparent part 6 of the housing 1 such that no edges or grooves are present on the transparent coating 53. A layer of a second coating may be applied on top of the coating 51, as shown at 52 on FIG. 3.

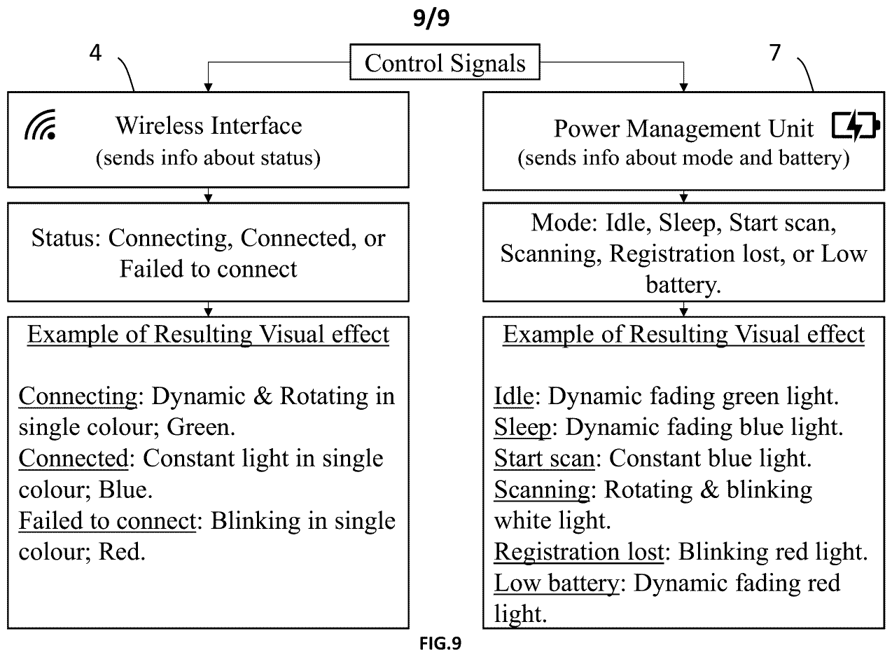

FIG. 9 shows a table with an overview of control signals from the wireless interface 4 and the power management unit 7 with information about connection status and scan mode, respectively. The power management unit 7 further provides information about the status of the battery 5. The figure shows that the wireless interface 4 sends a control signal to the processing unit 2 with information about the status of the wireless interface 4. The status of the wireless interface 4 may be one or more of the following:
a “connecting” status, indicating that the wireless interface 4 is connecting to an external device,
a “connected” status indicating that the wireless interface 4 is connected to the external device, and
a “failed to connect” status indicating that the wireless interface 4 failed to connect to the external device.

The figure further shows examples of resulting visual effects performed by the plurality of LEDs 3 as indication of a current status. For example, when the status is the “connecting status” then the plurality of LEDs 3 provide a rotational dynamic lighting effect with a single colour of spectra. For example, each of the plurality of LEDs 3 blinks such that a rotational appearance of the emitted light is visualized. The single colour in this example is green, but may be blue, green, white, or any other colour. In another example, when the status is the “connected status” then the plurality of LEDs 3 emit a light such that the appearance of the emitted light from the plurality of LEDs 3 seem solid or constant in a blue colour. In yet another example, when the status is the “failed to connect”, the plurality of LEDs 3 blink such that a dynamic appearance of the emitted light is visualized in a red colour.

FIG. 9 further shows that the power management unit 7 of the intraoral scanning device 10 sends a control signal to the processing unit 2 with information about the mode of the intraoral scanning device 10 and about the status of the battery 5. The mode of the intraoral scanning device 10 may be one or more of the following:
an “idle” mode indicating that the intraoral scanning device 10 is ready to perform a scan,
a “sleeping” mode indicating that the intraoral scanning device 10 is operating with a lower power consumption than in the idle mode,
a “start scan” mode indicating that the intraoral scanning device 10 is starting a scan session,
a “scanning” mode indicating that the intraoral scanning device 10 is in a scan session, and
a “registration lost” mode indicating that the intraoral scanning device 10 failed to process intraoral scan data captured by an image sensor of the intraoral scanning device 10.

The status of the battery 5 may be a “low battery” status indicating that the battery 5 should be recharged or exchanged with a new battery.

The figure further shows examples of resulting visual effects performed by the plurality of LEDs 3 as indication of a current mode or battery 5 status. For example, when the mode is the “idle” mode, then the plurality of LEDs 3 provide a dynamic fading lighting effect in the green colour. For example, each of the plurality of LEDs 3 blinks such that a dynamic fading appearance of the emitted light is visualized. In another example, when the mode is the “sleep” mode, then the plurality of LEDs 3 provide a dynamic fading lighting effect in the blue colour. In another example, when the mode is the “start scan” mode, then the plurality of LEDs 3 provide a constant blue light. In another example, when the mode is the “scanning” mode, then the plurality of LEDs 3 provide a rotating and blinking effect in white light. In another example, when the mode is the “registration lost” mode, then the plurality of LEDs 3 provide a blinking lighting effect in the red colour. In another example, when the status is the “low battery” status, then the plurality of LEDs 3 provide a dynamic fading lighting effect in the red colour.

Although some embodiments have been described and shown in detail, the disclosure is not restricted to such details, but may also be embodied in other ways within the scope of the subject matter defined in the following claims. In particular, it is to be understood that other embodiments may be utilized, and structural and functional modifications may be made without departing from the scope of the present invention.

Benefits, other advantages, and solutions to problems have been described herein with regard to specific embodiments. However, the benefits, advantages, solutions to problems, and any component(s)/unit(s) that may cause any benefit, advantage, or solution to occur or become more pronounced are not to be construed as critical, required, or essential features or components/elements of any or all the claims or the invention. The scope of the invention is accordingly to be limited by nothing other than the appended claims, in which reference to a component/unit/element in the singular is not intended to mean “one and only one” unless explicitly so stated, but rather “one or more.” A claim may refer to any of the preceding claims, and “any” is understood to mean “any one or more” of the preceding claims.

It is intended that the structural features of the devices described above, either in the detailed description and/or in the claims, may be combined with steps of the method, when appropriately substituted by a corresponding process.

As used, the singular forms “a,” “an,” and “the” are intended to include the plural forms as well (i.e., to have the meaning “at least one”), unless expressly stated otherwise. It will be further understood that the terms “includes,” “comprises,” “including,” and/or “comprising,” when used in this specification, specify the presence of stated features, integers, steps, operations, elements, and/or components, but do not preclude the presence or addition of one or more other features, integers, steps, operations, elements, components, and/or groups thereof. It will also be understood that when an element is referred to as being “connected” or “coupled” to another element, it can be directly connected or coupled to the other element, but an intervening element may also be present, unless expressly stated otherwise. Furthermore, “connected” or “coupled” as used herein may include wirelessly connected or coupled. As used herein, the term “and/or” includes any and all combinations of one or more of the associated listed items. The step of any disclosed method is not limited to the exact order stated herein, unless expressly stated otherwise.

It should be appreciated that reference throughout this specification to “one embodiment” or “an embodiment” or “an aspect” or features included as “may” means that a particular feature, structure or characteristic described in connection with the embodiment is included in at least one embodiment of the disclosure. Furthermore, the particular features, structures or characteristics may be combined as suitable in one or more embodiments of the disclosure. The previous description is provided to enable any person skilled in the art to practice the various aspects described herein. Various modifications to these aspects will be readily apparent to those skilled in the art, and the generic principles defined herein may be applied to other aspects.

The claims are not intended to be limited to the aspects shown herein but is to be accorded the full scope consistent with the language of the claims, wherein reference to an element in the singular is not intended to mean “one and only one” unless specifically so stated, but rather “one or more.” Unless specifically stated otherwise, the term “some” refers to one or more.

1. An intraoral scanning device for acquiring intraoral scan data from a three-dimensional dental object during a scanning session, the intraoral scanning device comprising:
a processing unit configured to process intraoral scan data of a patient and provide 2D image data and/or 3D image data;
a wireless interface configured to transmit the 2D image data and/or the 3D image data;
a battery configured to power at least the processing unit and the wireless interface;
a housing configured to accommodate at least the processing unit, the wireless interface and at least partially the battery; and
a plurality of LEDs accommodated by the housing, and the plurality of LEDs is configured to emit light through a transparent part of the housing made of a material that is configured to emit the light that passes through the material, and wherein the emitted light indicates a status of the battery and/or the wireless interface, and/or indicates a mode of the intraoral scanning device.

2. The intraoral scanning device according to claim 1, wherein the material of the transparent part is configured to diffuse the emitted light that passes through the material.

3. The intraoral scanning device, according to claim 1, wherein the transparent part of the housing is made of at least a single layered coating structure, and at least another part of the housing is made of a multiple layered coating structure, and wherein an outer layer of the multiple layered coating and the single layered coating comprise the same material.

4. The intraoral scanning device according to claim 1, wherein the plurality of LEDs is arranged circumferentially to the housing.

5. The intraoral scanning device according to claim 1, wherein the plurality of LEDs includes at least three different colour spectra.

6. The intraoral scanning device according to claim 1, wherein the processing unit is configured to generate at least one control signal to control power delivered to one or more of the plurality of LEDs.

7. The intraoral scanning device according to claim 6, wherein the processing unit is further configured to change at least the one control signal over time so that the plurality of LEDs emits at least one static or dynamic lighting effect.

8. The intraoral scanning device according to claim 5, comprising a user interface adapted to receive a user input to control operation of the processing unit so as to facilitate a change in the colour that is a combination of the at least three different colour spectra.

9. The intraoral scanning device according to claim 3, wherein the housing is made of a plastic shell, and the another part of the plastic shell that is coated with the multiple layered coating structure includes following layers;
a first layer including a base coating that minimizes light going into the second layer,
a second layer including a coloured layer, and
a third layer including a transparent coating.

10. The intraoral scanning device according to claim 3, wherein the housing is made of a plastic shell, and the transparent part of the plastic shell that is coated with the single layered coating structure includes a transparent coating.

11. The intraoral scanning device according to claim 9, wherein the first layer includes at least white and/or silver particles for scattering light that penetrates the plastic shell away from the second layer.

12. The intraoral scanning device according claim 1, wherein the housing includes a plastic shell that comprises a light guiding path configured to guide the light of the plurality of LEDs to the transparent part of the housing.

13. The intraoral scanning device according to claim 1, wherein the plurality of LEDs is arranged below the transparent part of the housing.

14. The intraoral scanning device according to claim 1, comprising a light blocker arranged on a first side of the plurality of LEDs preventing the emitted light from the plurality of LEDs to emerge on a front side of the plurality of LEDs.

15. The intraoral scanning device according to claim 1, comprising at least an optical sensor arranged in vicinity to the plurality of LEDs, and wherein a sensor light blocker is arranged between the optical sensor and the plurality of LEDs for preventing emitted light from the plurality of LEDs to be captured by the at least optical sensor.
